(19) United States
(12) Patent Application Publication
(10) Pub. No.: US 2026/0199671 A1
(43) Pub. Date: Jul. 16, 2026

(54) IONTOPHORETIC DRUG DELIVERY DEVICE

(71) Applicant: CAMBRIDGE ENTERPRISE LIMITED, Cambridge Cambridgeshire (GB)

(72) Inventors: Tobias Eduard NAEGELE, Cambridge Cambridgeshire (GB); George MALLIARAS, Cambridge Cambridgeshire (GB); Ulrich Felix KEYSER, Cambridge Cambridgeshire (GB)

(21) Appl. No.: 19/128,984

(22) PCT Filed: Nov. 10, 2023
(86) PCT No.: PCT/EP2023/081527

§ 371 Date: May 9, 2025

(30) Foreign Application Priority Data
Nov. 11, 2022 (EP) 22386080.0

(51) Int. Cl. A61N 1/30; A61N 1/04; C12M 1/42

(52) U.S. Cl. CPC A61N 1/303; A61N 1/0444; C12M 35/02

(57) ABSTRACT

The present invention relates to a drug delivery device, a method to transport a charged species, a method to manufacture a drug delivery device, and a method to generate a charged fluid. A drug delivery device is configured to deliver a drug to a drug delivery site in a bodily tissue, the drug delivery device comprising a cavity configured to continuously receive a charged fluid, wherein the drug delivery device is configured to receive a charged particle comprising a drug in a space adjacent to the cavity and drive the charged particle from the space to the drug delivery site, the charged particle being driven by the action of an electric field generated by the charged fluid in the cavity.

### FIELD OF THE INVENTION

The present invention relates to a drug delivery device, a method to transport a charged species, a method to manufacture a drug delivery device, and a method to generate a charged fluid.

### BACKGROUND

With more than 11,000 new cases each year in the UK, brain tumours are a common disease which significantly impacts the life of affected patients. On average, only 12% of patients survive for five or more years after the diagnosis. Depending on each individual case, brain tumours are treated by a combination of chemotherapy, radiotherapy and, if possible, surgical resection. A fundamental limitation for the success of chemotherapy is the blood-brain-barrier which plays a pivotal role in protecting the central nervous system from toxic substances but also significantly reduces the amount of cancer drugs which can be delivered into a tumour systemically. Additionally, bad vascularisation of solid brain tumours further complicates delivery of therapeutically relevant drug doses. Off-target toxicity of many common chemotherapeutics places strong constraints on the amount of drugs which can be safely systemically administered.

Convection enhanced delivery is a technique of pumping a drug fluid directly into a treatment site of a patient, such as a tumour. The quantity of a drug that can be safely delivered by convection enhanced delivery is limited by the local pressure build-up that inevitably occurs at the delivery site when the fluid is delivered. This technique also suffers from the problem of drug reflux.

Iontophoresis is the use of electric fields for the delivery of charged species. Iontophoresis is applicable in the field of drug delivery and is distinguished from convection-enhanced drug delivery in that iontophoresis is capable of delivering only a drug molecule or particle to the site of interest, without also needing to deliver the bulk fluid in which the drug is dissolved or suspended. Thus, delivery of drugs by iontophoresis can achieve a high drug concentration at a drug delivery site without drastically increasing local pressure at the site.

While iontophoresis has been used since the 1920s, implantable iontophoretic devices are relatively new. Notable work is published in the following documents, all of which are incorporated by reference herein in their entirety:
(1) EP 2 401 027 B1.
(2) J. D. Byrne et al. ‘Local iontophoretic administration of cytotoxic therapies to solid tumors’. In: Science Translational Medicine 7.273 (February 2015), 273ra14-273ra14; doi: 10.1126/scitranslmed.3009951.
(3) J. D. Byrne et al. ‘Iontophoretic device delivery for the localized treatment of pancreatic ductal adenocarcinoma’. In: Proceedings of the National Academy of Sciences 113.8 (February 2016), pp. 2200-2205; doi: 10.1073/pnas.1600421113.
(4) J. D. Byrne, J. J. Yeh and J. M. DeSimone. ‘Use of iontophoresis for the treatment of cancer’. In: Journal of Controlled Release 284 (August 2018), pp. 144-151; doi: 10.1016/j.jconrel.2018.06.020.
(5) T. A. Sjöström et al. ‘A Decade of Iontronic Delivery Devices’. In: Advanced Materials Technologies 3.5 (March 2018), p. 1700360; doi: 10.1002/admt.201700360.
(6) A. Williamson et al. ‘Controlling epileptiform activity with organic electronic ion pumps’. In: Advanced Materials 27.20 (2015), pp. 3138-3144; doi: 10.1002/adma.201500482.
(7) L. Waldherr et al. ‘Targeted Chemotherapy of Glioblastoma Spheroids with an Iontronic Pump’. In: Advanced Materials Technologies (April 2021), p. 2001302; doi: 10.1002/admt.202001302.

A fundamental challenge for iontophoresis is the creation of electric fields for extended periods of time because at perfectly blocking electrodes (i.e. electrodes where no electrochemical reactions are occurring), electrical double layers quickly form on the electrode surface which screen the electric field. As a result, with blocking electrodes drugs cannot be delivered. In traditional iontophoresis therefore, high voltages are applied which cause hydrolysis of water. While this reaction allows charge transfer, large amounts of corrosive chlorine gas form at the anode and hydrogen gas at the cathode, which can damage the device and can cause harm to the patient. Furthermore, the gas builds up in the delivery device and significantly reduces efficiency.

An alternative approach is using a solid electrode material which can be oxidised/reduced, such as Ag/AgCl or PEDOT:PSS. Ag⁺ however is toxic to the patient and both Ag/AgCl and PEDOT:PSS electrodes are depleted during use, and therefore limit how long the device can work.

Another limitation of existing drug delivery devices utilising an electrode is that the drug comes in contact with the electrode and can therefore itself undergo a redox reaction which might render it harmful. Depending on the applied voltage, any molecule coming into contact with the working electrode may undergo some form of electrochemical reaction. As a consequence, traditional iontophoretic devices will chemically change the drug molecules over time which can render them useless and/or harmful. The possible drug reactions will be very likely a severe issue when trying to get regulatory approval for a medical device.

Iontophoretic devices which have been published in literature may be grouped into two categories: traditional iontophoretic devices; and organic electronic ion pumps (OEIPs).

### Traditional Iontophoretic Devices

Traditional iontophoretic drug delivery devices have been used for transdermal delivery (i.e. delivery across the skin of a patient) of drugs. An example of a traditional iontophoretic device is shown schematically in FIG. 1.

The traditional iontophoretic device 100 comprises a reservoir 101 containing the drug molecule 102 and the working electrode 103. The reservoir is either open or sealed off using a membrane. The device 100 also comprises a counter electrode 104 placed elsewhere on the patient, far away from the working electrode 103. In the example of FIG. 1, an electric current (e.g. DC current) applied between the electrodes 103, 104 causes a positively-charged drug (D⁺) to be driven into the tissue (skin) of the patient in the vicinity of the anode 103 (working electrode). Anions A in the skin of the patient (such as Cl⁻ ions) are drawn to the anode 103, and cations C⁺ are drawn to the cathode 104 (counter electrode). These traditional devices are based on hydrolysis, i.e. the water in which the drug is dissolved is split on the electrodes as shown in the following electrochemical half-equations.

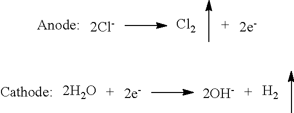

Due to these reactions, large amounts of corrosive chlorine gas form at the anode and hydrogen gas at the cathode. The gas formation on the electrodes causes significant problems as gas bubbles will accumulate in the device rendering the device inoperable after a while. Also, at both electrodes very corrosive species are formed: at the anode chlorine gas and at the cathode OH⁻, which will damage the device. This fundamentally limits the long term usefulness of the known devices.

Hydrolysis generally happens at both the working electrode (the anode: producing Cl₂ gas) and the counter electrode (the cathode: producing H₂ gas). In some arrangements, the working electrode can be the cathode, it depends on the charge of the drug which needs to be delivered. ‘Working electrode’ is used herein as the name for the electrode where the drug is repelled from.

### Organic Electronic Ion Pumps (OEIPs)

OEIPs have emerged more recently than traditional iontophoretic devices, and are very similar to the traditional iontophoretic devices. The only difference to traditional iontophoretic devices is that OEIPs use a solid film of the conducting polymer PEDOT:PSS as working electrode (anode). PEDOT:PSS can be oxidised and therefore allows to drive the device for a short time until the PEDOT:PSS film is depleted. Then, drug delivery stops. An advantage of this approach is that no gases are formed but a disadvantage is that the devices can be operated only for a relatively short time due to depletion of the electrodes. OEIPs are usually used for delivery of small amounts of molecules directly into cells over a short time period and thus are not suitable for many applications such as cancer drug delivery.

### SUMMARY OF THE INVENTION

In view of these problems, there is a need for devices and methods capable of safely delivering high concentrations of drugs to a delivery site, over long periods of time, without being subject to problems of off-target toxicity. Such devices and methods would be particularly useful in the treatment of cancerous tumours in the brain and elsewhere, and of various other diseases and conditions.

A first aspect of the present invention is a drug delivery device configured to deliver a drug to a drug delivery site in a bodily tissue, said drug delivery device comprising a cavity configured to continuously receive a charged fluid, wherein the drug delivery device is configured to: receive a charged particle comprising a drug in a space adjacent to the cavity; and drive the charged particle from the space to the drug delivery site, the charged particle being driven by the action of an electric field generated by the charged fluid in the cavity.

The charged fluid received in the cavity of the drug delivery device forms effectively a “fluid electrode”, which generates an electric field to interact with the charged particle comprising the drug in the space adjacent to the cavity. The charged fluid is continuously replenished and thus not subject to degradation in the way that a solid electrode directly in contact with a drug solution would be. An electric field may therefore be maintained for a long time, enabling long-term and stable delivery of the drug. The drug delivery device operates on the principles of iontophoresis, that is, using electric fields to drive the movement of the drug. The device is therefore capable of supplying a high concentration of the drug to the site of interest without causing local over-pressures. Furthermore, by providing the drug in a space adjacent to the cavity, rather than in the cavity with the charged fluid itself, detrimental reactions of the drug with other substances (such as the charged fluid, or any solid electrode that may be present inside the cavity) are prevented.

The charged particle comprising the drug may be the drug itself (i.e. a charged atom or molecule that is the drug to be delivered). Alternatively, the charged particle comprising the drug may be a particle (such as a nanoparticle) carrying the drug and other components. The charged particle comprising the drug may be a constituent of a drug fluid comprising other substances such as a solvent.

The cavity may be a channel configured to receive a continuous flow of the charged fluid. The charged fluid is a fluid (e.g. a liquid) that comprises a charged component, and thereby possesses as a whole a net or overall charge, which is either positive or negative in polarity. The cavity may comprise an elongate channel, said elongate channel configured to support a continuous flow of the fluid. The elongate channel is preferably shaped so as to facilitate a laminar flow profile therein. For instance, the elongate channel may have an aspect ratio that creates a laminar flow profile of fluid therein. For instance, the aspect ratio of the elongate channel may be at least 10, preferably at least 20, more preferably at least 30, more preferably at least 40, more preferably at least 50, more preferably at least 60, more preferably at least 70. Facilitating laminar flow in the cavity has the effect of minimising the formation of pockets of low concentration of the charged component of the charged fluid. Thus, substantially the entire content of the cavity may be continuously replenished.

Preferably, the space adjacent to the cavity is a drug channel configured to continuously receive the charged particle comprising the drug. In such an embodiment, the period of time over which the drug delivery device can deliver a drug to the drug delivery site of the patient is not limited by the size of the device itself. Rather, it is limited only by the length of time over which the drug and the charged fluid are supplied to the device. This therefore enables very long-term, theoretically endless, delivery of the drug to the patient. The drug channel may be or comprise an elongate channel. The elongate channel is preferably shaped so as to facilitate a laminar flow profile therein. For instance, the elongate channel may have an aspect ratio that creates a laminar flow profile of fluid therein. For instance, the aspect ratio of the elongate channel may be at least 10, preferably at least 20, more preferably at least 30, more preferably at least 40, more preferably at least 50, more preferably at least 60, more preferably at least 70. Facilitating laminar flow in the drug channel has the effect of minimising the formation of pockets of low concentration of the drug, which are more likely to form in a turbulent system. Thus, substantially the entire content of the drug channel may be continuously replenished.

Preferably, the drug delivery device comprises an electrode (i.e. a working electrode) in fluid contact with the cavity. The electrode in fluid contact with the cavity may be a solid electrode. The electrode is configured to provide a charge to a fluid so as to generate the charged fluid. That is, the charged fluid may be generated at the electrode by a charge-transfer interaction between the electrode and a feed fluid that initially does not carry an overall charge. In this embodiment, the position of the electrode relative to the cavity and the space adjacent to the cavity is not particularly limited. The electrode may be provided inside the cavity, and an uncharged fluid continuously supplied to the cavity, such that the charged fluid is continuously generated inside the drug delivery device. Additionally or alternatively, the electrode may be provided at a position upstream of the cavity, such that the fluid gains a charge prior to entry to the cavity, and the charged fluid is continuously supplied to the drug delivery device. In the case where the drug delivery device is configured to be implanted or partially implanted in a patient tissue, the electrode may be positioned inside the bodily tissue during use or outside the bodily tissue during use.

In use of the drug delivery device, a counter-electrode may be positioned elsewhere on the body of the subject to whom the drug is to be delivered, such that a voltage may be applied between the working electrode and the counter-electrode to drive the drug delivery.

Preferably, the drug delivery device comprises a first membrane between the cavity and the space adjacent to the cavity. The first membrane may selectively prevent any bulk fluid (such as solvent) from passing either way between the cavity and the space adjacent to the cavity. The first membrane may therefore advantageously prevent mixing of fluids between the different cavities which could otherwise lead to inefficiency or inefficacy of the device.

The first membrane may be configured to selectively allow a species having a charge of opposite polarity to that of the charged particle comprising the drug to pass into the cavity from the space adjacent to the cavity. This may enhance the driving of the drug to the drug delivery site by the electric field generated by the charged fluid in the cavity. The first membrane may additionally or alternatively be configured to selectively allow a species having a charge of the same polarity as that of the charged particle comprising the drug to pass into the cavity from the space adjacent to the cavity. The first membrane may be a non-selective membrane such as a porous membrane, which permits particles of either charge polarity (or no charge) to pass through it. Alternatively, the first membrane may be a selective membrane. The first membrane may be charge-selective in the sense that only particles of a certain charge polarity are permitted to pass through. The first membrane may be, for instance, an ion exchange membrane. When the first membrane is a selective membrane, such as an ion exchange membrane, the first membrane may prevent passage therethrough of certain species. For instance, the selectivity of the first membrane may prevent the charged particle comprising the drug from entering the cavity, and/or prevent charged species within the charged fluid from entering the space adjacent to the cavity. This selectivity is advantageous because it can prevent detrimental reactions that would otherwise occur, such as a reaction between the drug and the charged fluid, or between the drug and an electrode inside the cavity.

Preferably, the drug delivery device comprises a second membrane between the space adjacent to the cavity and the drug delivery site. The second membrane is configured to selectively allow the charged particle comprising the drug to pass to the drug delivery site from the space adjacent to the cavity. The action of the electric field generated by the charged fluid in the cavity drives the charged particle comprising the drug across the second membrane towards the drug delivery site. The second membrane may prevent passage of a bulk drug fluid from the space adjacent to the cavity to the drug delivery site. That is, when the space adjacent to the cavity contains a drug fluid that supports the charged particle comprising the drug (e.g. a solvent in which the a drug is dissolved, or a liquid in which the charged particles comprising the drug are suspended), the second membrane may selectively allow passage of only the charged particle comprising the drug to the drug delivery site, while retaining the remaining drug fluid in the space adjacent to the cavity. The second membrane thus enables iontophoretic delivery of the charged particle comprising the drug to the drug delivery site, while preventing the potentially harmful transfer of non-therapeutic fluid to the drug delivery site. The second membrane may be a non-selective membrane such as a porous membrane, or may be a selective membrane such as an ion exchange membrane.

A second aspect of the invention is a method to transport a charged species, said method comprising: continuously receiving a fluid in a cavity of a transport device, said fluid having or being provided with an electric charge so as to generate an electric field; receiving a charged species in a space adjacent to the cavity; and transporting the charged species away from the space, the charged species being transported by the action of said electric field.

Optionally, the charged species is transported in a direction away from the cavity in addition to being transported away from the space adjacent to the cavity.

A third aspect of the invention is a method of manufacturing a drug delivery device, said method comprising: providing a component having a cavity therein, the cavity configured to continuously receive a charged fluid; providing a first membrane between the cavity and a space adjacent to the cavity; and providing a second membrane in contact with the space adjacent to the cavity, the second membrane being configured to selectively allow a particle comprising a drug to pass out of the space adjacent to the cavity in a direction away from the cavity.

A fourth aspect of the invention is a drug delivery device comprising a fluid for delivering a drug to a drug delivery site, the fluid configured to: gain a charge from an electrode; and when charged, generate an electric field to drive a charged particle in a space adjacent to the fluid in a direction away from the fluid, the charged particle comprising the drug.

The fluid in the drug delivery device is preferably configured to gain a charge from a solid electrode. That is, the fluid includes a species that is able to undergo a charge transfer interaction with the electrode, thereby imparting to the fluid a net positive or negative charge. For instance, the fluid may have a composition including a species that is readily capable of being oxidised or reduced at the electrode, thereby facilitating ionic-electric charge transfer between the electrode and the fluid. Drug delivery by the drug delivery device is driven by this charge transfer interaction. The drug delivery device operates on the principles of iontophoresis, and is therefore capable of supplying a high concentration of the drug to the site of interest without causing local over-pressures. Furthermore, by providing the drug in a space adjacent to the cavity, rather than in the cavity with the charged fluid itself, detrimental reactions of the drug with other substances (such as the charged fluid, or any solid electrode that may be present inside the cavity) are prevented. The cavity may be channel configured to receive a continuous flow of the fluid, such as an elongate channel facilitating a laminar flow profile therein.

Preferably, the fluid is configured to gain the charge from the electrode without causing significant generation of a gas at the electrode. More preferably, the fluid is configured to gain the charge from the electrode without causing any generation of gas at the electrode. Traditional iontophoretic devices whose driving mechanism is a gas-generating reaction such as hydrolysis, are subject to the problems of poor longevity of the device due to release of (possibly corrosive) gases or other corrosive species, and harm to the patient due to the release of such gases inside the body. The drug delivery device of the present aspect does not rely on such gas-generating reactions to drive the iontophoretic delivery of drugs and thus is useful for safe long-term drug delivery to a patient tissue.

Optionally, the drug delivery device comprises the electrode that is configured to provide the fluid with the charge.

In use, a counter-electrode may be positioned elsewhere on the body of the subject to whom the drug is to be delivered, such that a voltage may be applied between the working electrode and the counter-electrode to drive the drug delivery.

Preferably, the drug delivery device is configured to continuously receive a flow of the fluid, wherein a flow path of the fluid contacts the electrode such that the fluid gains the charge when it passes the electrode. The fluid is thus continuously replenished and continuously provided with a charge by the electrode, and thus not subject to degradation in the way that a solid electrode directly in contact with a drug solution would be. An electric field may therefore be maintained for a long time, enabling long-term and stable delivery of the drug. Because the charge transfer interaction undergone by the fluid with the electrode does not cause significant (or any) generation of a gas, the continuous provision of a charge to the fluid does not cause detrimental gas build-up over time which would otherwise damage the device and/or cause harm to a patient if occurring inside the tissue.

Preferably, the drug delivery device comprises a fluid inlet for receipt of said fluid into said drug delivery device, and a fluid outlet for removal of said fluid from said drug delivery device. The drug delivery device may be configured to support continuous flow of fluid from the fluid inlet to the fluid outlet. For instance, the receipt and removal of fluid may be at the same volumetric rate so as to generate steady-state continuous flow from inlet to outlet.

Preferably, the drug delivery device comprises an elongate channel, said elongate channel configured to support a flow of the fluid. The elongate channel is preferably shaped so as to facilitate a laminar flow profile therein. For instance, the elongate channel may have an aspect ratio that creates a laminar flow profile of fluid therein. For instance, the aspect ratio of the elongate channel may be at least 10, preferably at least 20, more preferably at least 30, more preferably at least 40, more preferably at least 50, more preferably at least 60, more preferably at least 70. Facilitating laminar flow in the channel has the effect of minimising the formation of pockets of low concentration of charged components of the charged fluid. Thus, substantially the entire content of the channel may be continuously replenished.

A fifth aspect of the present invention is a method to transport a charged species, said method comprising: providing a fluid with an electric charge; positioning the charged species in a space adjacent to the fluid provided with the electric charge; and driving the charged species in a direction away from the fluid, by action of an electric field generated by the fluid.

A sixth aspect of the present invention is a drug delivery device comprising an electrode and a channel, the channel being in contact with the electrode and configured to receive a fluid, wherein: the electrode is formed of a double-sided piece of electrode material; and the channel is shaped so as to be in contact with two sides of the piece of electrode material.

The drug delivery device of the present aspect is configured to receive a fluid in a channel in contact with an electrode. Thereby, the fluid may undergo a charge transfer interaction with the surface of the electrode. The electrode can be a double-sided piece of electrode material. That is, the electrode can be formed of a piece of material having at least two sides (such as elongate sides) or surfaces on which charge transfer interactions can occur. The two sides can be opposite sides of a generally planar piece of material. For example, the two sides can be the top side and the bottom side of a planar piece of material. The shape of the channel in the device can be such that the fluid comes into contact with at least two sides of the electrode. The active surface area of the piece of electrode material in the device is thus advantageously increased, compared with a setup in which only one side of the electrode is active and in contact with the channel. This can improve the efficiency of the device, in that a greater rate of charge transfer is achieved for a given volume of electrode material. This may also reduce the size and/or cost of manufacture of a drug delivery device, in that for a given output a smaller volume of electrode material is required. The piece of electrode material may have further sides which are in contact with the channel. Also, the electrode may be formed of several pieces of electrode material, each of which has two or more sides in contact with the channel.

The drug delivery device of the sixth aspect may be suitable for iontophoretic drug delivery driven by charge supply to the fluid, in the same way as the drug delivery device of the first aspect or the drug delivery device of the fourth aspect.

Preferably, the channel is shaped so as to be in contact with two opposite sides of the double-sided piece of electrode material. The channel may therefore conform to the shape of the electrode material surface so as to advantageously maximise the surface area of the electrode in contact with the channel in which fluid is received for charge transfer. The channel preferably comprises an elongate channel. For instance, where the electrode has elongate sides, the channel may be formed of elongate portions that are in contact with respective elongate sides of the electrode. The elongate channel or elongate portions of the channel are preferably shaped so as to facilitate a laminar flow profile therein. For instance, the elongate channel or elongate portions of the channel may have an aspect ratio that creates a laminar flow profile of fluid therein. For instance, the aspect ratio of the elongate channel may be at least 10, preferably at least 20, more preferably at least 30, more preferably at least 40, more preferably at least 50, more preferably at least 60, more preferably at least 70. Facilitating laminar flow in the channel has the effect of minimising the formation of pockets of low concentration of charged components of the charged fluid. Thus, substantially the entire content of the channel may be continuously replenished.

Preferably, the channel is configured to receive a flow of the fluid. Preferably, the electrode is configured to provide the fluid with a charge as the fluid flows through the channel. Preferably, the channel is shaped such that a bulk flow direction of the fluid changes as the fluid flows past different sides of the double-sided piece of electrode material. The flow path of the fluid may therefore conform to the shape of the electrode surface and “wrap around” the electrode so as to advantageously maximise the surface area of the electrode in contact with the flow path of the fluid in the channel. This can maximise the amount of charge transfer occurring between the electrode and the flowing fluid for a given volume of electrode material. In one example, the direction of flow over one side of the electrode is substantially opposite to the direction of flow over the other side of the electrode.

Preferably, the channel comprises an elongate channel configured to support a flow of the fluid.

Preferably, the drug delivery device of the present aspect is configured to drive a charged particle from a space adjacent to the channel to a drug delivery site, the charged particle being driven by the action of an electric field generated by the charged fluid in the channel.

A seventh aspect of the invention is a method to generate a charged fluid, said method comprising: providing an electrode that is formed of a double-sided piece of electrode material; and flowing a fluid past the electrode, such that the flowing fluid comes into contact with two sides of the piece of electrode material and thereby receives a charge.

An eighth aspect of the invention is a fluid electrode comprising a cavity configured to continuously receive a charged fluid, wherein in use the charged fluid in the cavity generates an electric field that emanates from the cavity.

Preferably, the cavity is a channel configured to support a flow of the charged fluid, preferably wherein the channel comprises an elongate channel.

Preferably, the elongate channel has an aspect ratio that creates a laminar flow profile of the charged fluid therein. For instance, the aspect ratio of the elongate channel may be at least 10, preferably at least 20, more preferably at least 30, more preferably at least 40, more preferably at least 50, more preferably at least 60, more preferably at least 70.

Preferably, the fluid electrode comprises a fluid inlet for receipt of said fluid into said fluid electrode, and a fluid outlet for removal of said fluid from said fluid electrode.

Preferably, the fluid electrode comprises a first membrane in contact with the cavity, preferably wherein said first membrane is an ion exchange membrane.

Preferably, the fluid electrode comprises a second membrane, wherein the first membrane and the second membrane define therebetween a space adjacent to the cavity, wherein said space adjacent to the cavity is configured to receive a second charged fluid, said second charged fluid comprising a cell culture medium and/or a conductive electrolyte.

Preferably, the fluid electrode is configured to drive the second charged fluid from the space to a delivery site through the second membrane.

A ninth aspect of the invention is a method to generate an electric field in a medium, said method comprising: inserting a fluid electrode in the medium, the fluid electrode configured to continuously receive fluid; and continuously supplying a fluid to said channel, said fluid having or being provided with an electric charge, so as to generate an electric field emanating into said medium from said fluid electrode. The medium may contain cells, and the generated electric field may be applied to cause growth of said cells within said medium.

A tenth aspect of the invention is a method of generating a continuous electric field, the method comprising providing a fluid with a charge to generate a charged fluid, and continuously providing the charged fluid to a cavity in a device, such that the continuous electric field emanates from said cavity.

Any of the above aspects may incorporate features described herein in relation to any other of the aspects.

### BRIEF DESCRIPTION OF DRAWINGS

FIG. 1 depicts a traditional iontophoretic drug delivery device.

FIG. 2 depicts a drug delivery device according to an embodiment.

FIG. 3 depicts a charge-transfer interaction at an electrode that forms a charged fluid to be received in the drug delivery device of FIG. 2.

FIG. 4 depicts a cavity for continuously receiving a charged fluid, in a drug delivery device according to an embodiment.

FIG. 5 depicts a drug channel of a drug delivery device according to an embodiment.

FIG. 6 depicts a drug delivery device of a preferred embodiment.

FIG. 7 depicts a particular arrangement of a drug delivery device according to an embodiment.

FIG. 8 depicts a drug delivery system comprising a drug delivery device implanted in a patient.

FIG. 9 depicts a 3D model of a particular construction of a drug delivery device according to an embodiment.

FIG. 10 depicts a particular positioning of an electrode and membranes in a drug delivery device according to an embodiment.

FIG. 11 depicts another particular positioning of an electrode and membranes in a drug delivery device according to an embodiment.

FIG. 12 depicts a drug delivery device comprising an additional electrode according to an embodiment.

FIG. 13 depicts a drug delivery device having a channel in contact with two sides of a double-sided electrode, according to an embodiment.

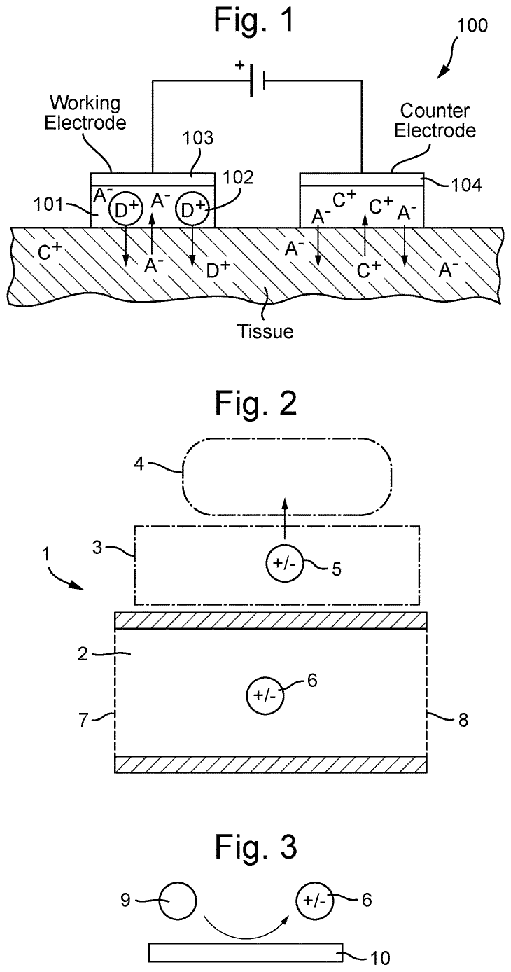

### DETAILED DESCRIPTION OF EMBODIMENTS

Exemplary embodiments will hereinafter be described with reference to the drawings.

The invention provides a novel way of local drug delivery into drug delivery sites such as brain tumours, for example for the treatment of cancer. Devices of the invention may be implanted directly into patient tissue such as directly into the brain tumour or surrounding tissue, and can deliver drugs directly to the delivery site.

FIG. 2 shows a schematic of a drug delivery device 1 according to an embodiment. The drug delivery device includes a cavity 2 configured to continuously receive a charged fluid. In use, a charged particle 5 comprising a drug is received in a space 3 adjacent to the cavity 2. The composition of the charged fluid is described further below, but in general the charged fluid comprises at least one charged component 6 such that the charged fluid in the cavity 2 has a net charge and thus generates an electric field which emanates from the cavity 2. The action of said electric field causes movement of the drug 5 from the space 3 towards a drug delivery site 4 in a bodily tissue.

In use, the drug delivery device 1 may be implanted in the bodily tissue close to the drug delivery site 4. The drug delivery site 4 may be a tumour.

The charged fluid is a fluid (e.g. a liquid) having a formulation which comprises at least one charged component 6, giving the fluid as a whole a net positive or negative charge. For instance, the charged fluid may be a solution of a charged particle (such as an ion) within a neutral solvent (such as water). The charged fluid may comprise any number of different charged components, as long as the fluid as a whole possesses a net charge. Preferably the charged fluid is biocompatible.

The cavity 2 is configured to continuously receive the charged fluid. The cavity 2 may be a channel that defines a flow path for continuous flow of the charged fluid. The channel may comprise an inlet 7 to continuously receive the charged fluid into the cavity, and may comprise an outlet 8 to continuously expel the charged fluid out of the cavity. The continuously-received charged fluid generates a continuous electric field whose action causes the charged particle 5 comprising the drug in the space 3 adjacent to the cavity 2 to move towards the drug delivery site 4, as described in more detail below. The continuously-replenished charged fluid in the cavity may be regarded as a “fluid electrode”, that is not subject to the usual problems of electrode degradation in traditional iontophoretic devices.

The charged fluid that is received in the cavity 2 of the drug delivery device 1 may have become charged by a charge transfer interaction of an initial “feed” fluid with an electrode. That is, a feed fluid having no overall charge or an overall charge different to that of the charged fluid, may be brought into contact with an electrode, such that at least one component of the feed fluid interacts with the electrode to gain or lose an amount of charge. FIG. 3 shows schematically an interaction of a component 9 present in the feed fluid with an electrode 10 which is a solid electrode, so as to generate the charged component 6 of the charged fluid. As a result of the charge transfer interaction, the net charge of the feed fluid changes, so that the feed fluid becomes the charged fluid comprising the charged component 9.

The charge transfer interaction between the component 9 of the feed fluid and the electrode 10 may be an electrochemical reaction such as oxidation or reduction, that occurs on the surface of the electrode 10. The interacting component 9 of the feed fluid may therefore be a species (referred to herein as a “redox species”) that is able to be oxidised or reduced. Preferably, the feed fluid comprises a redox species 9 whose reaction at the electrode surface has a large exchange current density, meaning that the redox species can be easily oxidised or reduced. As a result of the oxidation or reduction of the redox species 9 in the feed fluid, the charge of the redox species 9 changes so that the redox species 9 becomes the charged component 6, thereby causing the net charge of the feed fluid to change as it contacts the electrode 10, and thereby converting the feed fluid into the charged fluid.

Preferably, the redox species is biocompatible. While in the ideal case the redox species will never get in contact with the patient, it might not always be possible to be avoided completely, and so biocompatibility provides an advantage.

Preferably, the component 9 of the feed fluid which interacts with the electrode 10 to form the charged component 6, undergoes this charge-transfer interaction (such as oxidation or reduction when the component 9 is a redox species) without causing significant generation of a gas at the electrode. Most preferably, the interaction causes no generation of gas at all at the electrode. The driving mechanism of the drug delivery device is thus a charge-transfer interaction that does not cause build-up inside the device or inside a patient in use, which would otherwise damage the device or reduce its efficiency, or cause harm to the patient. The drug delivery device 1 is thus suitable for safe long-term drug delivery to a patient tissue.

A preferred redox species is ferrocyanide, which can be oxidised to form ferricyanide by the following reaction:

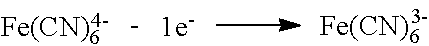

This electrochemical reaction converts electric charge (i.e. electrons) into ions creating an electric field which repulses positively charged drug molecules or positively charged particles comprising drug molecules in the drug channel, and drives them into the patient's tissue. Ferrocyanide is a preferred redox species in the present invention because its oxidation to form ferricyanide does not cause the formation of a gas. This can improve the safety and efficiency of a drug delivery device. Another suitable redox species is ascorbic acid, which is capable of being oxidised.

When the charged fluid is generated from a feed fluid as described above, the generation of the charged fluid from the feed fluid may occur inside the drug delivery device 1, such as inside the cavity 2, or may occur outside the drug delivery device 1. That is, the cavity 2 can be configured to continuously receive the charged fluid by continuous generation of the charged fluid at an electrode 10 inside the cavity, and/or by continuous supply of the charged fluid in an already charged state to the cavity 2 from elsewhere. When the charged fluid is continuously supplied to the cavity in an already charged state, the charged fluid may have been generated at an electrode 10 which is outside the cavity 2, such as outside the drug delivery device 1 entirely.

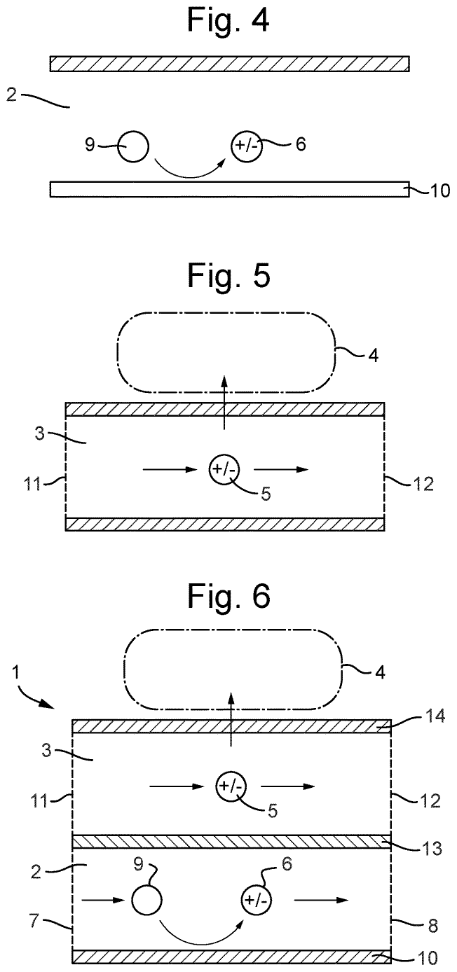

FIG. 4 shows the cavity 2 of a drug delivery device. As shown, an electrode 10 is in fluid contact with the cavity 2. The position of the electrode 10 is not particularly limited as discussed further below, but in general the electrode 10 may be positioned inside the cavity 2 itself (e.g. forming a wall of the cavity as shown in FIG. 4), or may be positioned outside the cavity 2 yet in contact with a flow path of fluid that subsequently enters the cavity 2 after contact with the electrode 10. The charge transfer between the electrode 10 and the feed fluid (e.g. between the electrode and the redox species 9) occurs continuously such that the charged fluid is continuously generated. The continuous generation of the charged fluid in this way may occur inside the cavity 2 itself (when the electrode is positioned as shown in FIG. 4), or may occur in the flow path of fluid preceding the cavity. In the embodiment of FIG. 4, the feed fluid is flushed continuously past the electrode 10 in order to continuously replenish the supply of the species (e.g. redox species 9) that undergoes the charge transfer at the electrode 10. The electrode 10 is illustrated as a solid electrode comprised of an electrically conducting solid material, such as a metal.

The drug delivery device 1 can be configured to receive a charged particle 5 comprising a drug, in a space 3 adjacent to the cavity 2, as shown in FIG. 2. The charged particle 5 may be a constituent of a drug fluid that is received by the drug delivery device 1. That is, the charged particle 5 comprising the drug may be dissolved or suspended in a drug fluid that also comprises other components, such as a liquid solvent. The composition of the drug fluid is not particularly limited, as long as it comprises the charged particle 5 which itself comprises the drug.

The composition or form of the charged particle 5 itself is also not particularly limited, as long as it carries a charge. The charged particle 5 may itself be a charged drug molecule, such as the chemotherapy drug cisplatin. That is, the charged particle comprising the drug may be the drug itself. Alternatively, the charged particle 5 may comprise one or more drug molecules as well as other components. For instance, the charged particle 5 could be a carrier vehicle of a scale larger than molecular (such as a nanoparticle), which carries one or more drug molecules. In the latter case, the drug molecules themselves do not necessarily need to have a charge, as long as the carrier vehicle has an overall charge. This therefore enables the delivery of uncharged drugs.

A particular application of the present invention is in the delivery of the positively-charged cancer drug cisplatin. Other cancer drugs of any charge can also be delivered. Examples of cancer drugs deliverable by the devices and methods disclosed herein include: cisplatin; carboplatin; oxaliplatin; doxorubicin; gemcitabine; temozolomide; bleomycin; fluorouracil; and irinotecan. The devices and methods disclosed herein are not limited to the delivery of cancer drugs, and it is possible to deliver other charged drug molecules with it targeting different diseases. Examples of other drugs deliverable by the devices and methods disclosed herein include neurotransmitters such as gamma-aminobutyric acid (GABA), and local anaesthetics such as lidocaine. The devices and methods disclosed herein may also be used for delivery of any of the drugs disclosed in ‘Use of iontophoresis for the treatment of cancer’ (J. D. Byrne, J. J. Yeh and J. M. DeSimone, Journal of Controlled Release 284 (August 2018), pp. 144-151, doi: 10.1016/j.jconrel.2018.06.020) and ‘Selected Medicines Used in Iontophoresis’ (T. M. Karpinski, Pharmaceutics 2018, 10, 204; doi: 10.3390/pharmaceutics10040204).

The space 3 adjacent to the cavity 2 may be a channel (“drug channel”) configured to continuously receive the charged particle 5. For instance, the drug channel may receive a continuous flow of a drug fluid comprising the charged particle 5 which itself comprises the drug. FIG. 5 illustrates the drug channel 3 and drug delivery site 4. The drug channel 3 may have a drug channel inlet 11 to continuously receive the charged particle 5 into the drug channel 3. The continuously-received charged particle 5 is continuously driven by action of the electric field from the charged fluid in the cavity, towards the drug delivery site 4. The drug channel 3 may further comprise a drug channel outlet 12 configured to continuously expel from the drug channel the remaining components of the drug fluid after an amount (e.g. all) of the drug has been driven from the drug channel 3 to the drug delivery site 4. Those remaining components may include a solvent, and may include a quantity of the charged particle 5 which has not been driven to the drug delivery site 4.

The drug delivery device preferably comprises one or more membranes. A preferred embodiment comprising a first membrane 13 and a second membrane 14 is shown in FIG. 6. The properties and function of these membranes are described further below. In the embodiment of FIG. 6, the cavity 2 is a channel configured to receive a continuous flow of the charged fluid, an electrode 10 is in fluid contact with the cavity so as to generate the charged fluid (comprising charged component 6) continuously from a feed fluid as described above, and the space 3 adjacent to the cavity is a drug channel configured to receive a continuous flow of the charged particle 5 comprising the drug. However, the provision of membranes such as the first membrane 13 and/or second membrane 14 is also possible in any of the other arrangements of the drug delivery device described herein.

The drug delivery device 1 may comprise a first membrane 13 disposed between the cavity 2 and the space 3 adjacent to the cavity. The first membrane 13 separates the cavity 2 from the space 3 adjacent to the cavity, thereby preventing or reducing mixing of fluids contained in the separate regions. This may advantageously prevent undesired chemical reactions from occurring, such as reactions between drugs contained in the space 3 and the electrode 10, which may otherwise cause the drugs to become useless or even harmful to a patient.

The first membrane 13 may be a porous membrane that is non-selective to the charge polarity of particles that are allowed to cross the first membrane 13. Alternatively, the first membrane 13 may a selective membrane which only allows particles having a certain charge polarity to pass through. For instance, the first membrane 13 may be an ion exchange membrane only allowing a certain polarity of ions to pass through (either an anion exchange membrane only allowing passage of anions, or a cation exchange membrane only allowing passage of cations).

The first membrane 13 may be configured to allow a species having a charge of opposite polarity to that of the charged particle 5 to pass into the cavity from the space adjacent to the cavity. In this embodiment, the movement of such species across the first membrane 13 may enhance the driving of the charged particle 5 comprising the drug towards the drug delivery site 4 by the electric field generated by the charged fluid in the cavity. That is, the action of the electric field generated by the charged fluid in the cavity 2 attracts species (e.g. species present in a drug fluid of which the charged particle 5 is a constituent) having a charge opposite to that of the charged particle 5 to enter the cavity from the space adjacent to the cavity, through the first membrane 13. A charge imbalance is thereby created in the space adjacent to the cavity, driving the movement of the charged particle 5 away from the space and towards the drug delivery site.

The first membrane can be a selective membrane (e.g. an ion exchange membrane) that allows only passage of species having a charge of opposite polarity to that of the charged particle 5, and prevents the passage of species having a charge of the same polarity as that of the charged particle 5. For instance, when the charged particle 5 comprising the drug is positively charged (such as the positively-charged drug molecule cisplatin), the first membrane 13 may be an anion exchange membrane. When the charged particle is negatively charged, the first membrane 13 may be a cation exchange membrane.

The first membrane 13 may be configured to allow a species having a charge of the same polarity to that of the charged particle 5 to pass into the cavity from the space adjacent to the cavity. In a particular embodiment, the first membrane is a selective membrane (e.g. an ion exchange membrane) that allows passage only of species having a charge of the same polarity as that of the charged particle 5, and prevents the passage of species having a charge of opposite polarity to that of the charged particle 5.

The drug delivery device 1 may comprise a second membrane 14 disposed between the space 3 adjacent to the cavity and the drug delivery site 4. The second membrane 13 separates the space 3 from the drug delivery site, thereby preventing or reducing mixing of fluids contained in the separate regions. This may advantageously prevent external fluids from entering the drug delivery device which may harm the efficiency of the device. This may also advantageously prevent bulk fluid in the space 3 (such as a drug fluid) from entering the drug delivery site 4, as described further below.

The second membrane 14 is configured to allow the charged particle 5 comprising the drug to pass to the drug delivery site from the space adjacent to the cavity. The charged particle 5 comprising the drug therefore passes through the second membrane 14 in use of the drug delivery device 1, due to the action of the electric field generated by the charged fluid in the cavity 2.

The second membrane 14 may be configured to allow passage only of the charged particle 5 comprising the drug, while preventing passage of bulk fluid. As described herein, the charged particle 5 comprising the drug may be a constituent of a drug fluid in the space 3 adjacent to the cavity 2. The second membrane 14 may therefore prevent the constituents of that drug fluid other than the charged particle 5 comprising the drug from passing to the drug delivery site 4. In this way, the second membrane advantageously allows the drug delivery device 1 to deliver the drug to the drug delivery site 4 without causing local build-up of pressure at the drug delivery site due to the presence of excess fluid.

The second membrane 14 may be non-selective to the charge polarity of particles allowed to pass through, such as a porous membrane. Alternatively, the second membrane 14 may be a selective membrane (such as an ion exchange membrane) that allows particles only of a certain charge polarity to pass through. For instance, the second membrane 14 may configured to only allow particles having the charge polarity of the charged particle 5 to pass through, while preventing passage of particles of opposite charge. For example, when the charged particle 5 is the positively-charged drug molecule cisplatin, the second membrane 14 may be a cation exchange membrane that prevents the passage of negatively-charged ions.

In a preferred embodiment of the drug delivery device 1, a porous membrane is used as the second membrane 14 to separate the drug channel 3 from the drug delivery site 4 in patient tissue, and an ion exchange membrane is used as the first membrane 13 to separate the cavity 2 from the drug channel 3. However, as described above, it is also possible to use a non-selective porous membrane for the separation of the cavity and drug channel 3 or an ion exchange membrane to separate the drug channel 3 and the drug delivery site 4.

Examples of suitable membranes include Astom Neosepta AMX anion ion exchange membrane (particularly suitable for use as the first membrane 13 separating the drug channel 3 and cavity 2) and Repligen Spectra/Por 3 standard regenerated cellulose membrane 3.5 kD (particularly suitable for use as the second membrane 14 separating the drug channel 3 and the drug delivery site 4).

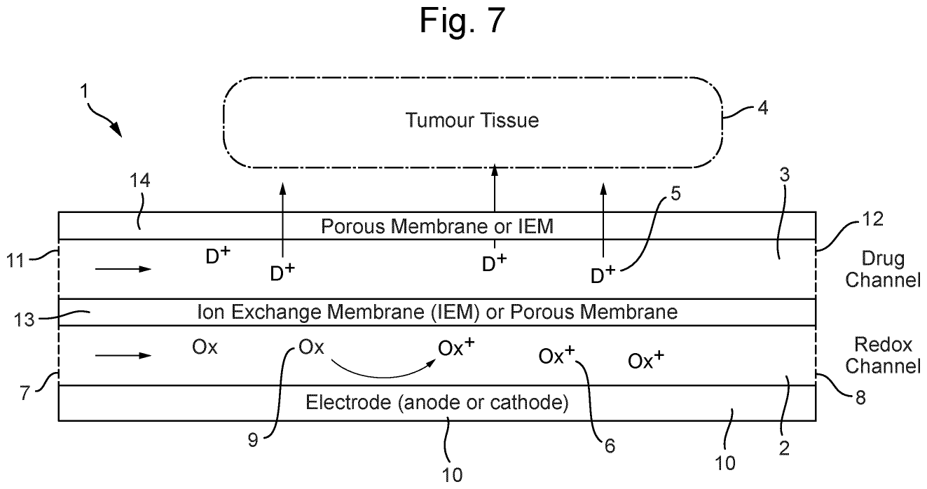

FIG. 7 shows a particular arrangement of the drug delivery device 1. The device 1 is essentially a two-channel microfluidic flow cell. Through the first channel called “redox channel” (being an example of the cavity 2), flows an aqueous solution of a redox species 9, being a molecule which can be easily oxidised. The redox channel 2 is in contact with an electrode 10 which may be made of a noble metal (e.g. Pt) or graphite. Adjacent to the redox channel is a drug channel 3, and an aqueous solution of the drug to be delivered flows through the drug channel 3. The redox channel 2 and drug channel 3 are separated from each other by a first membrane 13 that is an anion-exchange membrane. An anion-exchange membrane is a membrane which only allows anions to pass through and which blocks cations. The anion-exchange membrane 13 allows electric current to flow from the electrode 10 through both channels while it prevents the solutions in the two channels from mixing by convection which would have a detrimental effect on device performance. The drug channel 3 is separated from the tissue of the patient by a second membrane 14 which can be, for instance, a porous membrane or an ion exchange membrane. The second membrane 14 prevents pressure-driven flow of the bulk drug solution into the tissue.

In the embodiment of FIG. 7, the cavity 2 is a channel (“redox channel”), and contains the charged fluid. The charged fluid comprises positively charged component 6, which gains its charge by oxidation of a redox species 9 at an electrode 10 in fluid contact with the redox channel 2. The redox channel 2 is separate from the drug channel 3 which is a second microfluidic channel containing a drug solution (i.e. a drug fluid that is a solution of a charged drug molecule 5). The redox channel 2 and drug channel 3 are separated by an anion exchange membrane 13 (i.e. the membrane only allows anions to pass and blocks cations). Due to the oxidation of the redox species 9, a positive charge imbalance is created in the redox channel 2, which attracts negatively-charged particles (such as Cl ions) from the drug channel 3. Negatively charged particles from the drug channel 3 move into the redox channel 2. As a result, there is also a positive charge imbalance in the drug channel 3 formed which drives the positively-charged drug molecules 5 (which in this embodiment are cationic drug molecules, D⁺) through the second (outer) membrane 14 toward the drug delivery site 4 (which is depicted as tumour tissue). The ion-exchange membrane 13 prevents cations to be driven from the redox channel 2 into the drug channel 3 which would compete with the cationic drug 5 (e.g. cancer drug). The drug channel 3 is separated from the tumour tissue 4 by a second membrane 14 that can be a porous membrane or an ion exchange membrane. Continuous flow of the drug solution in the drug channel 3 allows to maintain a large drug concentration in the device 1. The device 1 has four fluidic ports: a redox channel inlet 7, a redox channel outlet 8, and a drug channel inlet 11, and a drug channel outlet 12.

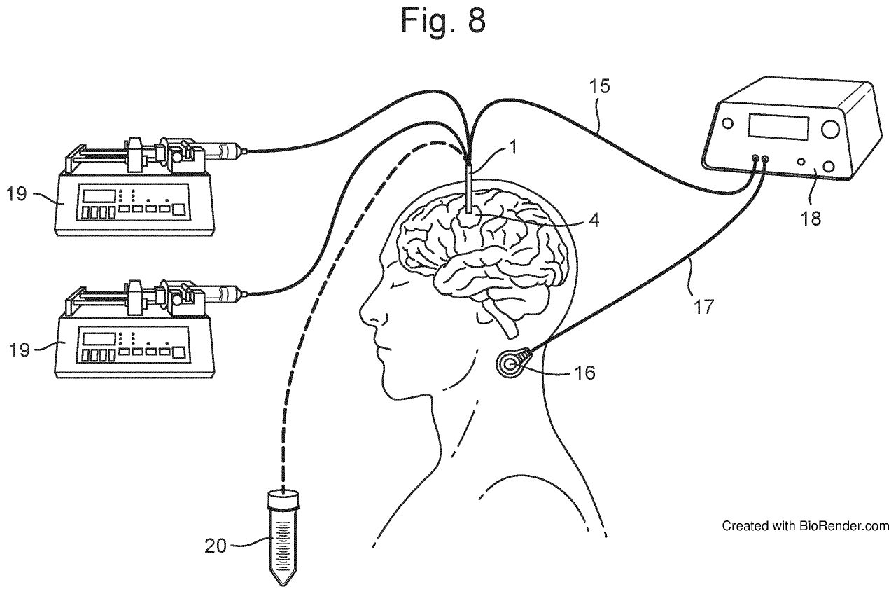

FIG. 8 shows an example of how the drug delivery device 1 such as that shown in FIG. 7, can be connected to a patient. In this example, the drug delivery site 4 is a brain tumour. An electrode cable 15 may connect the electrode 10 inside the device 1 to a power supply 18. In the arrangement shown, the electrode 10 is inside the device 1, however as described herein the electrode 10 is not limited to being inside the device 1 and could be outside the device 1, such as outside the patient tissue entirely. A counter-electrode 16 may be attached to the patient's skin. The counter-electrode 16 may be a commercial ECG electrode such as an Ag/AgCl electrode. The counter electrode 16 can be connected to power supply 18 by a counter-electrode cable 17. A voltage is applied between the electrode 10 and the counter-electrode 16, which drives the charge-transfer interaction (for instance, the oxidation or reduction of a redox species) to form the charged fluid received in the device 1. The inlets of the redox and drug channels may be connected to a microfluidic flow controller or a syringe driver 19. The outlets of the redox and drug channels can be connected to a waste container 20. The drug delivery device 1 can be implanted into the brain tumour 4 through a cranial window. A commercial port system can be used with the device (e.g. Renishaw Neuroinfuse).

The fluid in the redox channel 2 that gains a charge by interaction (e.g. oxidation or reduction) with the electrode 10 may be known as a “fluid electrode”. When the redox channel 2 contains a liquid solution of a redox species, it may be known as a “liquid electrode”. The concept of using a fluid electrode such as a liquid electrode has numerous advantages, for instance: it can prevent the formation of corrosive gases which is a major problem with traditional iontophoretic devices using hydrolysis as driving mechanism; it prevents the depletion of the working electrode as it is the case with the ‘organic electronic ion pumps’ allowing to run the device theoretically forever provided the redox solution is constantly supplied; it allows to separate the drug molecule from the working electrode preventing reactions of the drug molecule on the electrode surface; and it allows to drive the device at high currents, i.e. high drug delivery rates.

An example of a liquid electrode employs the redox species ferrocyanide. When a voltage is applied between the electrode and the counter-electrode, ferrocyanide gets oxidised to ferricyanide, driving the movement of the drug to the drug delivery site as described herein.

The devices and methods disclosed herein are not limited by the charge polarity of the charged particle 5 comprising the drug, or of the charged fluid. While FIG. 7 depicts a positively-charged fluid in the redox channel 2 (gaining a positive charge from electrode 10) creating an electric field which drives delivery of a positively-charged drug 5, the principles are equally applicable to negatively-charged fluids and drugs. This would mean inverting the polarity such that the working electrode 10 in the device becomes the cathode instead of the anode and that the sacrificial redox species would be reduced instead of oxidised.

The material from which the electrode 10 is made is not particularly limited. The drug delivery device 1 described herein can operate with any conducting electrode but the reaction rate and therefore the efficiency of the device is dependent on the electrode material. The electrodes can be made of any materials which are commonly used for electrochemical applications. Particular examples of suitable electrode materials include metallic electrodes (such as gold, platinum, platinum-iridium, stainless steel etc) and non-metallic carbon-based electrodes such as graphite.

As discussed herein, the electrode 10 may be inside the drug delivery device 1, such as forming a wall of the cavity 2 as shown in FIG. 7. The electrode 10 may also be placed completely outside of the drug delivery device 1, which is advantageous for applications where it is not desired to have the electrode 10 in the patient body. The electrode 10 is generally in fluid contact with the cavity 2, meaning that the charge-transfer interaction that generates the charged fluid may occur inside the cavity itself or at a point on the flow path of the fluid undergoing said interaction that is earlier than the cavity 2.

When the cavity 2 is a channel configured to receive a flow of the charged fluid, the electrode 10 may be placed in said channel at substantially the same point on the flow path of charged fluid as the first membrane 13. Such an arrangement is shown schematically in FIG. 7, which depicts the electrode 10 located directly underneath the membrane stack of the first membrane 13 and second membrane 14.

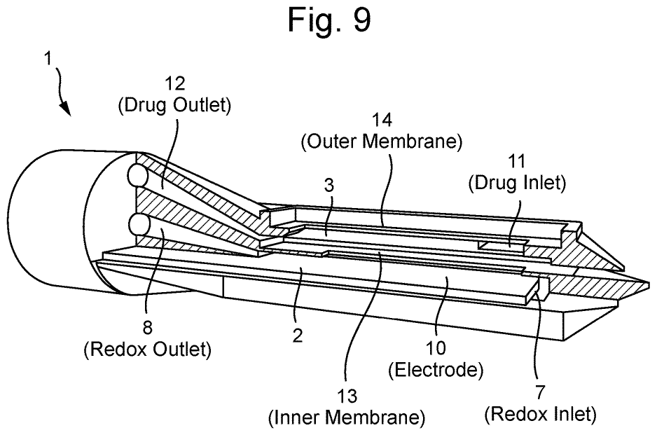

A 3D model of a particular example of the construction of such a drug delivery device is shown in FIG. 9.

FIG. 9 shows a drug delivery device 1 comprising a cavity 2 which in this embodiment is a channel comprising inlet 7 and outlet 8. Electrode 10 is shown in contact with the channel 2. First membrane 13 is disposed between the channel 2 and drug channel 3, which has a drug channel inlet 11 and drug channel outlet 12. Second membrane 14 is in contact with the drug channel 3. In use, a feed fluid may continuously enter the inlet 7 and become charged by continuous contact with electrode 10 to generate a charged fluid, which constitutes a so-called “fluid electrode”. A drug fluid containing a charged particle which comprises a drug, may be introduced continuously to the drug channel 3. By the mechanism described herein, the charged particle can be driven across the second membrane to a drug delivery site outside the drug delivery device 1. The drug delivery device 1 is particularly suitable for implantation in a patient body such as implantation in a brain tumour as shown in FIG. 8.

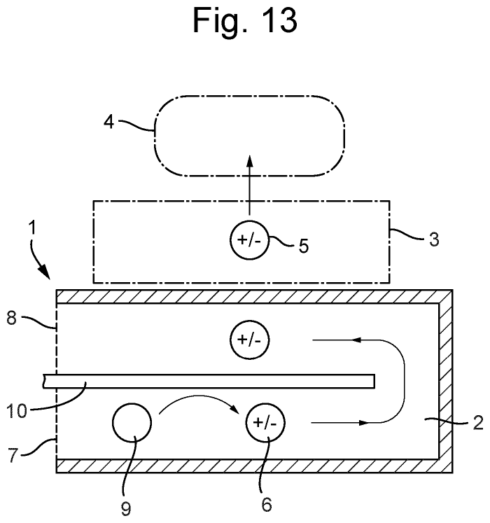

The device shown in FIG. 9 has a double sided electrode, explained in further detail below with respect to FIG. 13.

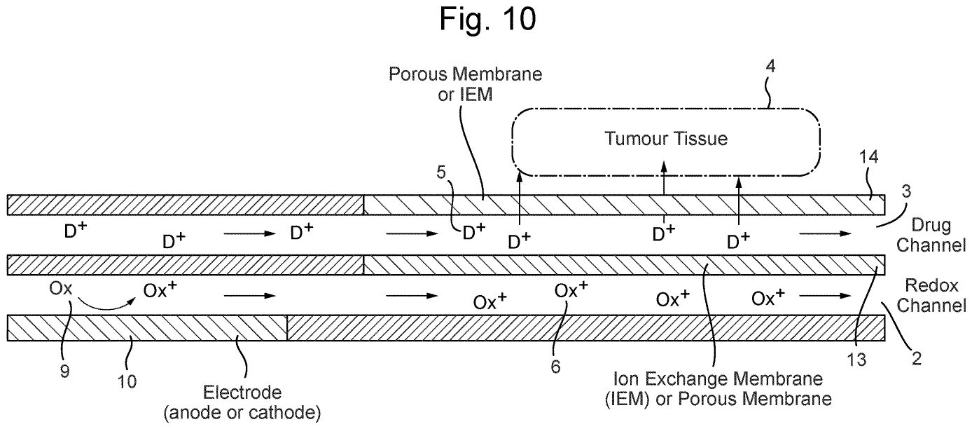

The position of the electrode 10 in relation to the membranes of the drug delivery device 1 in use is however not particularly limited. That is, the electrode 10 does not need to be located directly underneath the membrane stack, and can be shifted in position. FIG. 10 shows an alternative arrangement with the electrode being in a different position to that of FIGS. 7 and 9. In the arrangement of FIG. 10, the electrode 10 is placed in a position in the redox channel 2 that is upstream of the position of the first membrane 13, in terms of the flow path of the charged fluid. In this arrangement, the conversion of the feed fluid to the charged fluid by the electrode 10 occurs at a position earlier in the fluid flow path than the position at which the driving of the drug by the electric field occurs. This may give more flexibility to the device geometry in certain applications.

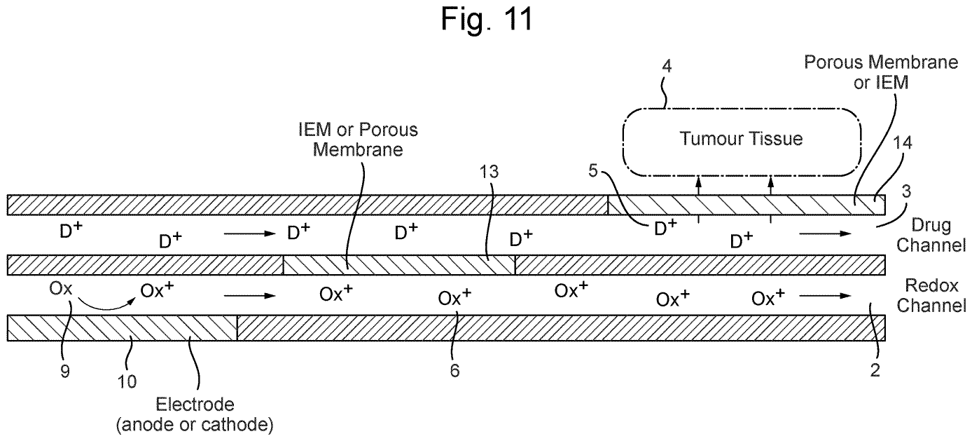

Additionally, also the first membrane 13 separating the redox channel 2 and the drug channel 3 does not need to be located directly underneath the second (outer) membrane 14 or directly above the working electrode 10. FIG. 11 shows an arrangement in which the redox channel 2 and drug channel 3 run parallel and adjacent to one another, and the first membrane 13 and second membrane 14 are positioned at different points on the parallel flow paths of the two channels. In the arrangement shown, the movement of particles of opposite charge polarity to the charged particle 5 containing the drug from the drug channel 3 to the redox channel 2 occurs earlier in the flow paths than the movement of the charged particle 5 from the drug channel 3 to the drug delivery site 4. This gives for certain applications more flexibility for the device geometry.

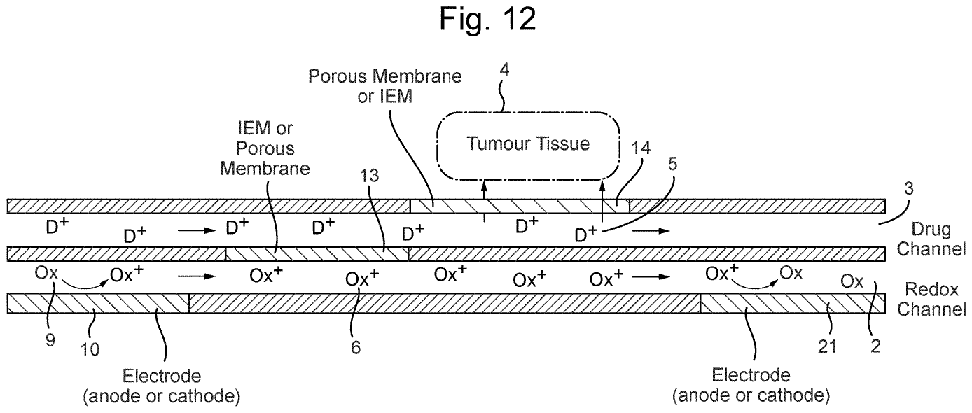

Another different geometry of the drug delivery device 1 is a three-electrode system as shown in FIG. 12. In this arrangement, where a second electrode 21 sits in the redox channel 2 which performs the opposite charge-transfer interaction as that of the working (“first”) electrode 10, so as to return the charged component 6 of the charged fluid back to its original state before it received a charge from the working electrode. For instance, the second electrode reduces the charged component 6 (such as ferricyanide) back to the redox species 9 (such as ferrocyanide). In the arrangement shown, the redox species 9 in the redox channel 2 gets oxidised on the working electrode 10 (which is an anode), passes the first membrane 13 which separates redox channel 2 and the drug channel 3, and then gets reduced back into its original state on the second electrode 21 (which is a cathode). This allows a circular flow of the redox species (i.e. the redox channel could be a closed system) where the redox species is repeatedly oxidised and reduced. The counter electrode 16 on the skin of the patient will ensure that drug molecules are still delivered. Thus, the fluid constituting the fluid electrode can be continuously recycled so as to minimise wasted material in use of the drug delivery device.

The fluid constituting the “fluid electrode” in the drug delivery device 1 (that is, the fluid that generates an electric field so as to drive the drug to the desired site) may have distinct characteristics that improve the efficiency, efficacy and safety of such devices. Thus, a further embodiment of the invention is a drug delivery device comprising a fluid for delivering a drug to a drug delivery site, the fluid configured to: gain a charge from an electrode; and when charged, generate an electric field to drive a charged particle in a space adjacent to the fluid in a direction away from the fluid, the charged particle comprising the drug. Said drug delivery device may correspond with the drug delivery device 1 of any of the other embodiments described herein. The electrode from which the fluid gains a charge may be the solid, working electrode 10 as described herein.

The fluid gains the charge from the electrode by a charge-transfer interaction of a component in the fluid with the electrode. That is, the fluid comprises a component capable of undergoing a charge-transfer such as a redox reaction, with the electrode, such that the charge of that component changes and the fluid as a whole gains an overall or net charge. Said fluid is preferably configured to gain the charge from the electrode without causing significant generation of a gas at the surface of said electrode. This makes the drug delivery device of this embodiment suitable for long-term efficient use and safe for the patient, because harmful build-up of gases is prevented which would otherwise damage or inhibit the device and cause harm to the patient due to pressure build-up and the possibility of released gases being corrosive or toxic. This property of the fluid also distinguishes it from fluids involved in traditional iontophoretic devices that are absolutely reliant on gas-generating reactions such as hydrolysis to drive the movement of particles such as drugs. Most preferably, the fluid is configured to gain the charge from the electrode without causing any generation of a gas at the electrode.

Suitable examples of the fluid in the drug delivery device are fluids containing ferrocyanide, and fluids containing ascorbic acid. Both of these molecules are capable of being oxidised so as to cause the fluid in which they are contained to become charged. The molecules of the fluid which undergo a charge-transfer interaction to give the fluid a charge may be referred to as “redox species”. The drug delivery device may comprise the electrode that is configured to provide the fluid with the charge, or the electrode may be placed outside the drug delivery device such that the charge transfer interaction occurs outside the device. The drug delivery device may be configured to continuously receive a flow of the fluid, and the flow path of the fluid may contact the electrode at a certain point such that the fluid gains the charge continuously as it passes the electrode. Continuously replenishing the fluid constituting the “fluid electrode” ensures that the drug delivery device can continuously deliver a drug over long periods of time because the problem of electrode deterioration that exists in traditional transport devices is overcome.

When the drug delivery device has an electrode in contact with the cavity for generation of the charged fluid, said electrode and cavity may have distinct geometric characteristics that improve the efficiency and efficacy of such devices. Thus, a further embodiment of the invention is a drug delivery device comprising an electrode and a channel, the channel being in contact with the electrode and configured to receive a fluid, wherein: the electrode is formed of a double-sided piece of electrode material; and the channel is shaped so as to be in contact with two sides of the piece of electrode material. This drug delivery device may correspond with the drug delivery device 1 of any of the other embodiments described herein. Said channel corresponds with the cavity 2 of the drug delivery device 1 as described in previous embodiments. A drug delivery device in accordance with this embodiment is shown schematically in FIG. 13 and also in FIG. 9.

FIG. 13 depicts a drug delivery device 1 including a channel 2 configured to receive a fluid, and an electrode 10 in contact with the cavity 2. The fluid received in the channel 2 is a fluid comprising a component 9 able to interact with electrode 10 to generate a charged component 6. The electrode 10 in this embodiment is formed of a double-sided piece of electrode material. The electrode material is not particularly limited and could be metallic (such as gold, platinum, platinum-iridium, stainless steel etc) or a non-metallic carbon-based electrode material such as graphite. The piece is double-sided, meaning it has at least two active sides or surfaces on which charge-transfer interactions with the fluid can take place. This arrangement advantageously maximises the active surface area of an electrode for a given volume of electrode material. This is distinct from an arrangement in which an electrode is positioned such that one side of the electrode is a surface in contact with the channel and able to undergo charge-transfer interactions, and the other side (e.g. the opposite side) is in contact with the casing of the drug delivery device. In the example depicted in FIG. 13, the piece of electrode material constituting the electrode 10 has two elongate sides both in contact with the channel 2, and the channel 2 is shaped such that fluid therein flows past both elongate sides of the electrode 10.

Maximising the active surface area of the electrode 10 in this way in turn maximises the chance of a particular component 9 in the fluid coming into contact with electrode 10 and becoming a charged component 6, thereby maximising the amount of charge that the fluid as a whole gains as it flows through the channel 2. As a result, when the charge of the charged fluid is used to drive movement of charged particles 5 (such as drug particles) in a space 3 adjacent to the cavity, the strength of the electric field emanating from the fluid in the channel 2 (the “fluid electrode”) is maximised so that the charged particles 5 are driven efficiently. The configuration of channel 2 and electrode 10 shown in FIG. 13 may therefore be used in combination with the drug delivery device 1 of any of the preceding embodiments described herein.

It is also desirable to design the channel 2 and electrode 10 of such a drug delivery device in a way that efficiently uses space, so that the device itself can be effective yet compact and thus suitable for implantation. Thus, in some arrangements, the channel 2 may be shaped so as to be in contact with two opposite sides of the double-sided piece of electrode material, as is the case in FIG. 13. Such an arrangement allows efficient use of space because the channel 2 can essentially surround the electrode 10. In the example of FIG. 13, fluid in the channel 2 flows past one side of the electrode 10, then flows past the opposite side of electrode 10 while driving movement of particles 5 in the adjacent space 3. In some arrangements, the channel 2 is configured to receive a flow of the fluid, and the channel is shaped such that a bulk flow direction of the fluid changes as the fluid flows past different sides of the double-sided piece of electrode material. This is also the case in FIG. 13. This allows continuous replenishing of the fluid which constitutes the “fluid electrode”, and also maximises efficiency of use of electrode material and the use of space, by shaping the channel so as to “wrap around” the surfaces of the electrode 10. A compact yet effective device for generating a charged fluid to deliver a drug may therefore be provided. In such embodiments, the electrode defines the flow channel of the fluid, i.e. is a structural part of the device, and/or the electrode may sit in a slot within a component which defines the flow path.

The arrangement of the channel 2 and electrode 10 shown in FIG. 13 enables efficient use of space and of electrode material for generating a charged fluid. The charged fluid may be used in drug delivery by the mechanisms described herein, for instance by further incorporating one or more membranes such as first membrane 13 and second membrane 14 in the device 1. The charged fluid may also be used for other purposes, having been generated efficiently by the arrangement shown.

A drug delivery device as shown in FIG. 13 may be applied in a similar way to any of the other drug delivery devices described herein. That is, the drug delivery device is preferably configured to drive a charged particle from a space adjacent to the channel to a drug delivery site, the charged particle comprising a drug, and the charged particle being driven by the action of an electric field generated by the charged fluid in the channel.

In any of the embodiments described herein, where the cavity 2 defining the fluid electrode is a channel, as depicted in FIGS. 6-7 and 9-13, said channel 2 is preferably an elongate channel (that is, the dimension of the channel in the direction of flow through the channel is relatively long compared with the dimensions perpendicular to the direction of flow). In embodiments where the channel 2 defining the fluid electrode involves a change in overall flow direction, such as the embodiment shown in FIG. 13, the channel is preferably formed of distinct elongate channel portions each of which are elongate (e.g. where each elongate channel portion contacts a different elongate side of the electrode 10). By making the channel 2 or portions of the channel 2 thin and elongated, laminar flow is encouraged within the channel 2.

Likewise, in any of the embodiments described herein, where the space 3 adjacent to the cavity is a drug channel, as depicted in FIGS. 5-7 and 9-12, said drug channel is preferably an elongate channel. This encourages laminar flow within the drug channel 3.

By facilitating continuous laminar flow in the channels of the device, substantially the entire content of the channels is continuously replenished, and formation of pockets of low concentration of drug (in drug channel 3) or redox mediator (in channel 2) is minimised or eliminated.

The elongate form of the channel 2 and/or drug channel 3 may be expressed in terms of an aspect ratio, that is, the ratio of channel length (in the direction of flow) to channel thickness (perpendicular to flow). More specifically, the aspect ratio of an elongate channel may be defined as the ratio of the length of the channel in the direction of bulk flow therethrough to the smallest dimension of the channel in a direction perpendicular to the bulk flow direction. Thus, for a channel of rectangular cross-section with length 12 mm, height 1.8 mm and width 150μ, the aspect ratio is the ratio of length to width (as this is smaller than height), so the aspect ratio is 80.

In some embodiments, the aspect ratio of the elongate channel 2, in which the fluid electrode is defined, is at least 10, preferably at least 20, more preferably at least 30, more preferably at least 40, more preferably at least 50, more preferably at least 60, more preferably at least 70, most preferably about 80. In some embodiments, the aspect ratio of the elongate drug channel 3 (or of each elongate portion of the drug channel 3) is at least 10, preferably at least 20, more preferably at least 30, more preferably at least 40, more preferably at least 50, more preferably at least 60, more preferably at least 70. A highly preferable aspect ratio is about 80. Example dimensions of a channel or channel portion applicable either to the cavity 2 or space adjacent the cavity 3 are: about 12 mm in length in the direction of flow; about 150 μm in thickness in one orthogonal dimension; and about 1.8 mm in width in the other orthogonal dimension.

In the various embodiments of the drug delivery device 1 described herein, the drug delivery device 1 uses electric fields to deliver charged drugs (being either drug molecules themselves, or carrier particles carrying drugs). This technique can deliver selectively only the drug molecules without any solvent (iontophoresis). This allows to achieve high cancer drug concentrations within the brain tumour without increasing the cranial pressure (creating local over-pressures) which is an important advantage over direct injection of cancer drugs into tumours (convection enhanced delivery). Further advantages of this approach are: that the drug does not come in contact with the electrode and therefore does not undergo electrochemical reactions; that no gaseous reaction side products are produced in the device; and that the device could be operated theoretically infinitely long with continuous supply of the charged fluid (or of the redox species).

The devices described herein are suitable for the delivery of drugs (delivered as drug molecules alone or carried on carrier vehicles) as described above. However, devices having substantially the same arrangement as the drug delivery device 1 may also be configured to deliver any charged particle, not necessarily comprising a drug. That is, the principles of the present invention enable the delivery of any charged molecule, atom or vehicle particle loaded with any substance. The present invention therefore provides, in addition to drug delivery devices, methods of transporting charged species in general and methods of generating charged fluids.

For instance, the present invention also provides a method to transport a charged species, the method comprising: continuously receiving a fluid in a cavity of a transport device, the fluid having or being provided with an electric charge so as to generate an electric field; receiving a charged species in a space adjacent to the cavity; and transporting the charged species away from the space, the charged species being transported by the action of said electric field. The method may be implemented using a device similar to the drug delivery device 1 of any of the embodiments herein. For instance, the charged species may be received continuously in the space adjacent the cavity, which itself may be a channel. The transporting the charged species away from the space may involve movement of the charged species across a second membrane 14, which may be a selective membrane, towards a delivery site. The transport may also involve movement of various species across a first membrane 13, which may be a selective membrane, between the cavity of the transport device and the space adjacent the cavity. It will be appreciated that such a method may include steps analogous to any one of the functional features described herein in relation to drug delivery devices.

The present invention similarly provides a method to transport a charged species, the method comprising: providing a fluid with an electric charge, without generating a gas; positioning the charged species in a space adjacent to the fluid that is provided with the electric charge; and driving the charged species in a direction away from the fluid, by action of an electric field generated by the fluid. The method may also be implemented using devices similar to the drug delivery device 1 of any of the embodiments herein. Thus, it will similarly be appreciated that such a method may include steps analogous to any one of the functional features described herein in relation to drug delivery devices.

In any of the methods to transport a charged species described herein, the charged species to be transported is not particularly limited. For instance, such a method could be used to dose substances into in vitro cell cultures in an electrically controlled fashion, i.e. as a tool for microbiologists.

The present invention similarly provides a method to generate a charged fluid, the method comprising: providing an electrode that is formed of a double-sided piece of electrode material; and flowing a fluid past the electrode, such that the flowing fluid comes into contact with two sides of the piece of electrode material and thereby receives a charge. Such a method could be implemented using a cavity 2 and electrode 10 as described herein in relation to various embodiments of drug delivery devices 1. Thus, it will be appreciated that said method may include steps analogous to any one of the functional features described herein in relation to drug delivery devices.

The present invention further provides a method of manufacturing a drug delivery device, the method comprising: providing a component having a cavity therein, the cavity configured to continuously receive a charged fluid; providing a first membrane between the cavity and a space adjacent to the cavity; and providing a second membrane in contact with the space adjacent to the cavity, the second membrane being configured to selectively allow a particle comprising a drug to pass out of the space adjacent to the cavity in a direction away from the cavity. The so-manufactured drug delivery device may be the drug delivery device 1 of any of the embodiments described herein, and it will be appreciated that the method of manufacturing the drug delivery device may involve providing or creating any combination of the device features described herein in relation to drug delivery devices.

Also, because the devices and methods described herein are able to create constant DC electric fields in a patient body, the technology has other applications beyond delivery of drugs or other particles. For instance, a “fluid electrode” or “liquid electrode” having any of the properties described or depicted herein may itself form an implant that generates a continuous electric field inside the body. Such an implant could be used for various applications including the enhancement of cell growth. For instance, such an implant could be used for facilitating growth of neurons in a certain direction, thereby assisting in recovery of nerve damage. Such a device may be similarly implanted into an cell culture outside of any body, and used for facilitating growth of cells in vitro.

The effect of AC electric fields on cell growth has been extensively studied in the last years. However, commonly used platinum, stainless steel or PEDOT:PSS electrodes do not allow to measure the effect of low frequency (i.e. <1 kHz or even DC) fields on cell growth due to the onset of hydrolysis of water and the associated gas formation and pH drift on the electrode surfaces at low frequencies. A redox flow iontophoretic device as described herein however is capable of creating low frequency or even DC fields in electrolytes without causing hydrolysis of water. Therefore, not only can the devices described herein be used as a medical device to facilitate nerve growth and regeneration, but the devices and methods described herein could be used as a tool to study the effect of low frequency electric fields on cell growth in vitro.

For creating low frequency electric fields, the following experimental geometries are possible:
two drug delivery devices of the present invention placed opposite to each other with the membranes 13 facing each other, in the case of DC field creation one device being the anode and the other the cathode;
one drug delivery device and the counter electrode being a conventional electrode such as Ag/AgCl, platinum or stainless steel (the conventional counter electrode would obviously suffer from the effects described above).

In such applications, because drug delivery is not the intention, the drug channel(s) 3 of the delivery device(s) need not be supplied with a drug, and could either be filled with a cell culture medium or a conductive electrolyte such a saline, or the drug channels 3 and outer 14 membranes could be omitted altogether leaving only the redox channel 2 and one single membrane 13 (e.g. ion exchange membrane).

Thus, a further aspect is a fluid electrode comprising a cavity configured to continuously receive a charged fluid (that is, a fluid having or being provided with an electric charge), wherein in use the charged fluid in the cavity generates an electric field (e.g. a continuous electric field) that emanates from the cavity. The fluid electrode of this aspect may be equivalent to that described herein in relation to the drug delivery device 1, except that it does not require the provision of a charged drug or other particle into a space adjacent to the cavity. Preferably, the fluid electrode incorporates any of the properties as described herein in relation to the “cavity” or “channel” of a drug delivery device. It is thus preferable for the fluid electrode to incorporate a channel, preferably elongate, which supports a flow of the charged fluid. As for the fluid channels of the above-described drug delivery device, it is preferable for the aspect ratio of the fluid electrode to encourage therein laminar flow. It is preferable that continuous flow is facilitated through the provision of a fluid inlet and a fluid outlet. The fluid electrode may be used as a transport device or controlled dosing device for any substance, and as such is not limited to being a drug delivery device.

1-35. (canceled)

36. A fluid electrode, comprising:
a cavity configured to continuously receive a fluid, said fluid having or being provided with an electric charge,
wherein, in use, the charged fluid in the cavity generates an electric field that emanates from the cavity.

37. The fluid electrode of claim 1, wherein the cavity is a channel configured to support a flow of the charged fluid, preferably wherein the channel comprises an elongate channel.

38. The fluid electrode of claim 2, wherein the elongate channel has an aspect ratio that creates a laminar flow profile of the charged fluid therein.

39. The fluid electrode of claim 3, wherein the aspect ratio of the elongate channel is at least 10, preferably at least 20, more preferably at least 30, more preferably at least 40, more preferably at least 50, more preferably at least 60, more preferably at least 70.

40. The fluid electrode of claim 1, comprising a fluid inlet for receipt of said fluid into said fluid electrode, and a fluid outlet for removal of said fluid from said fluid electrode.

41. The fluid electrode of claim 1, further comprising a first membrane in contact with the cavity, preferably wherein said first membrane is an ion exchange membrane.

42. The fluid electrode of claim 6, further comprising a second membrane, wherein the first membrane and the second membrane define therebetween a space adjacent to the cavity, wherein said space adjacent to the cavity is configured to receive a second charged fluid, said second charged fluid comprising a cell culture medium and/or a conductive electrolyte.

43. The fluid electrode of claim 7, configured to drive the second charged fluid from the space to a delivery site through the second membrane.

44. A drug delivery device configured to deliver a drug to a drug delivery site in a bodily tissue, said drug delivery device comprising the fluid electrode of claim 1, wherein the drug delivery device is configured to:
receive a charged particle comprising a drug in a space adjacent to the cavity; and
drive the charged particle from the space to the drug delivery site, the charged particle being driven by the action of an electric field generated by the charged fluid in the cavity.

45. The drug delivery device of claim 9, wherein the space adjacent to the cavity is a drug channel configured to continuously receive the charged particle.

46. The drug delivery device of claim 9, further comprising an electrode in fluid contact with the cavity, the electrode configured to provide a charge to a fluid so as to generate the charged fluid,
optionally wherein the electrode is positioned outside of the bodily tissue during delivery of the charged particle to the drug delivery site.

47. The drug delivery device of claim 9, further comprising a first membrane between the cavity and the space adjacent to the cavity, optionally wherein
the first membrane is configured to selectively allow a species having a charge of opposite polarity to that of the charged particle to pass into the cavity from the space adjacent to the cavity; and/or
the first membrane is configured to selectively allow a species having a charge of the same polarity as that of the charged particle to pass into the cavity from the space adjacent to the cavity.

48. The drug delivery device of claim 9, further comprising a second membrane between the space adjacent to the cavity and the drug delivery site, wherein the second membrane is configured to selectively allow the charged particle comprising the drug to pass to the drug delivery site from the space adjacent to the cavity.

49. A drug delivery device comprising a fluid for delivering a drug to a drug delivery site, the fluid configured to:
gain a charge from an electrode; and
when charged, generate an electric field to drive a charged particle in a space adjacent to the fluid in a direction away from the fluid, the charged particle comprising the drug.

50. The drug delivery device of claim 14, wherein the fluid is configured to gain the charge from the electrode without causing significant generation of a gas at the electrode.

51. The drug delivery device of claim 14, wherein
the drug delivery device comprises the electrode that is configured to provide the fluid with the charge; and/or
the drug delivery device is configured to continuously receive a flow of the fluid, wherein a flow path of the fluid contacts the electrode such that the fluid gains the charge when it passes the electrode.

52. A drug delivery device comprising an electrode and a channel, the channel being in contact with the electrode and configured to receive a fluid, wherein:
the electrode is formed of a double-sided piece of electrode material; and
the channel is shaped so as to be in contact with the two sides of the piece of electrode material.

53. The drug delivery device of claim 17, wherein the channel is shaped so as to be in contact with two opposite sides of the double-sided piece of electrode material.

54. The drug delivery device of claim 17, wherein the channel is shaped such that a bulk flow direction of the fluid changes as the fluid flows past different sides of the double-sided piece of electrode material.

55. The drug delivery device of claim 17, wherein the electrode is configured to provide the fluid with a charge as the fluid flows through the channel.
